(19) United States
(12) Patent Application Publication
(10) Pub. No.: US 2003/0073932 A1
(43) Pub. Date: Apr. 17, 2003

(54) Body fluid sample preparation

(76) Inventor: Mark Varey, York (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/236,075

(22) Filed: Sep. 5, 2002

Related U.S. Application Data
(60) Provisional application No. 60/317,477, filed on Sep. 5, 2001.

(51) Int. Cl. A61B 5/00

(52) U.S. Cl. 600/573

(57) ABSTRACT

The invention relates to a method for the isolation of soluble components from body fluid samples containing cellular material comprising the steps of contacting a sample with a porous support material; allowing said sample to dry to said support; optionally storing the support containing the sample prior to extraction of the soluble components from the insoluble cellular material; a body fluid sample collection device and a kit comprising components used in the above method.

### RELATED APPLICATIONS

This application claims the benefit under 35 U.S.C. § 119(e) of U.S. provisional application No. 60/317,477, filed Sep. 5, 2001, the entire disclosure of which is incorporated herein by reference.

### FIELD OF THE INVENTION

The invention relates to a method for the sampling of body fluid samples, for example, blood, semen, or lymph, typically for the diagnostic testing of the presence of biomolecules in said samples.

### BACKGROUND OF THE INVENTION

The preparation and/or sampling of bodily fluids is often a pre-requisite for many assays conducted on body fluid samples. Typically, separation methods involve chromatographic separation of particulate material from soluble material in a sample. This can often also include additional chromatographic steps to further purify the desired components from a sample.

A number of techniques involving the analysis of biomolecules (e.g. proteins, amino acids, DNA, RNA, intermediary metabolites), in body fluid samples are known. These range from basic wet chemistry methods which simply indicate the presence or absence of the biomolecule to relatively complex methods which involve sampling followed by separation, identification and quantitation of biomolecules which maybe present at very low levels. These methods typically involve the separation of fluid components using electrophoretic or chromatographic techniques followed by detection of the biomolecule(s). Examples of these techniques are disclosed in U.S. Pat. No. 3,637,489 which describes a process for the separation of blood components using a technique referred to as steric chromatography, also referred to as gel chromatography, exclusion chromatography or gel permeation chromatography. The method uses a porous substance into which the blood sample permeates at different rates dependent on the size of the components. A similar methodology is employed in U.S. Pat. No. 3,657,116 wherein separation of blood components from blood plasma or serum is achieved using a chromatographic column filled with porous glass particles.

In blood collection procedures samples of whole blood are typically collected from a patient by venous puncture through a cannula or needle attached to a syringe or collection tube. The samples are then shipped to the laboratory were the samples are processed for analysis.

A problem associated with prior art methods for the preparation of body fluid samples is that the prior art methods involve a number of chromatographic steps followed by selective absorption and elution from a solid or semi-solid support material. Inevitably the inclusion of a number of steps in the isolation procedure means that the practitioner obtaining the sample has to remove a greater volume of sample than is actually needed for the assay to take account of sample loss during processing. In addition, or alternatively, prior art methods which do not involve separation techniques are often not volumetric and may result in an insufficient sample being provided to conduct the assay.

It would be desirable to provide a method which reduces the number of steps used in the sample isolation procedure thereby minimising loss of trace components in said blood sample and therefore minimising the size of the sample obtained from the patient.

Moreover, it would also be desirable to provide a method of sample collection which is volumetric and reduces the likelihood of an insufficient sample being obtained from a patient. Furthermore it is becoming common for patients to provide samples for testing which are obtained in the home therefore removing the need for the patient to visit a doctors surgery or hospital. Body fluid samples, for example blood, are obtained by the patient by simply pricking the surface of a finger or thumb. In this case it is important that an adequate sample is taken to ensure a reliable test is provided.

### SUMMARY OF THE INVENTION

According to a first aspect of the invention there is provided a method for the collection of body fluid samples comprising the steps of:

i) contacting a body fluid sample with a porous support material;

ii) allowing said sample to dry to said porous support; optionally

iii) storing said porous support material containing said sample; and

iv) extracting from said support the sample retained in said support.

In a preferred method of the invention said body fluid sample is selected from: blood; semen; lymph fluid; cerebrospinal fluid; synovial fluid; tears; sweat; urine; saliva; or bone marrow.

In a further preferred method of the invention said porous support material is selected from: polyester; cellulose acetate; polyolefin; nylon.

In a yet further preferred method of the invention said porous support material is polyester.

In a further preferred method of the invention said support material is coated with a non-ionic surfactant, preferably a non-ionic detergent. Preferably said detergent is selected from Triton X100 or Tween 20.

In a yet further preferred method of the invention said support material is coated with at least about 0.05% w/w surfactant. More preferably said support material is coated with no more than about 2.0% w/w surfactant. More preferably still said support material is coated with about 1% w/w surfactant.

In a further preferred method of the invention said body fluid sample contains an immunoglobulin. Preferably said immunoglobulin type is selected from the following Ig isotypes: IgA, IgM, IgD, IgE and IgG (IgG comprises four sub-classes based on differences in the H chains, i.e. IgG1, IgG2, IgG3 and IgG4).

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (K or k), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxyl-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, δ, ε, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

In a further preferred method of the invention the extraction of soluble components from said support utilises a high ionic strength buffer. A high ionic strength extraction buffer preferably has an ionic strength greater than about 0.5M, more preferably greater than about 1.0M, still more preferably greater than about 1.5M, yet more preferably greater than about 2.0M, and most preferably greater than about 2.5M. An example of high ionic strength extraction buffer useful in accordance with the invention is provided below. Preferably said buffer includes a detergent.

According to a further aspect of the invention there is provided a body fluid sample collection device comprising: a porous sample collection part and associated therewith a handhold to facilitate sample collection.

In a preferred embodiment of the invention said sample collection part is manufactured from at least one of the following materials: polyester; cellulose acetate; polyolefin; nylon. More preferably still said part is manufactured from polyester.

In a further preferred embodiment of the invention said sample collection part comprises an elongate member which serves as a wick to facilitate sample collection. The elongate member can have any cross-sectional shape for example cylindrical, oval, square, rectangular, trapezoidal. Typically the wick has a substantially circular cross-sectional shape.

In a further preferred embodiment of the invention said handhold is integral with said wick.

In an alternative preferred embodiment said handhold is adapted to fit about said wick. Typically the adaptation may take the form of a member with a throughbore through which the elongate member passes. The throughbore is adapted to slidably co-operate with the elongate member. The handhold may be manufactured from the same material as the elongate member or from other suitable material, for example plastic.

The handhold enables easy manipulation of the wick when obtaining a fluid sample and prevents contamination of the sample and/or the practitioner obtaining the sample. Moreover, if the sample is to be obtained by the patient at home then the handhold assists the patient in obtaining a sample. It will be apparent to one skilled in the art that the obtaining of, for example, a blood sample by a patient, (as opposed to a medical practitioner), can cause difficulty for the patient.

In a further preferred embodiment of the invention the wick is dimensioned to obtain an aliquot of bodily fluid sufficient to conduct at least one assay.

In a further preferred embodiment of the invention said sample collection device is sterile. In a yet further preferred embodiment of the invention said porous sample collection part is provided with a surfactant coating. Preferably said coating is a detergent coating. More preferably said detergent coating is provided by Triton X100 or Tween 20.

According to a yet further aspect of the invention there is provided a body fluid sample collection kit comprising: porous support material; a storage container for said support; optionally a skin puncture device; and further optionally, antiseptic wipes and/or adhesive dressing.

In a preferred embodiment of the kit said porous support material is the sample collection device according to any previous aspect or embodiment of the invention.

In a further preferred embodiment of the kit said skin puncture device is a blade, lancet or needle. Preferably said skin puncture device is a needle which is provided with actuation means to propel the needle through the patients skin.

In a further preferred embodiment of the kit said storage container is a vial or similar device which is provided with a cap. Preferably said cap is a screw top cap. Preferably the cap is provided with a seal to prevent contamination of the sample contained therein.

In a further preferred embodiment the kit is contained with a container. Preferably said container is sealed to protect the kit components.

Preferably said kit is for the isolation of blood samples.

The kit is suitably adapted for safe transportation of the sample to the test centre for extraction of the soluble body fluid components from the porous support followed by analysis.

An embodiment of the invention will now be described, by example only, and with reference to the following figures:

### BRIEF DESCRIPTION OF THE DRAWINGS

FIG. 1 illustrates blood sample collection using the components of the sample collection kit; FIG. 1a, skin puncture using the pressure actuated needle; FIG. 1b, aliquot of blood from puncture wound; FIG. 1c, application of sample collection device; FIG. 1d, sample collection part fills by capillary action; and FIG. 1e, placing sample collection device in container.

### DETAILED DESCRIPTION OF THE INVENTION

### Body Fluid Sample Collection Kit

The body fluid sample collection kit comprises a number of components. The sample collection device (1) typically consists of two parts; a first part (2) which serves to provide a wick of porous material which is adapted to absorb a sample of bodily fluid (i.e. blood, semen, tears, lymph, sweat, urine, saliva, bone marrow). The wick is manufactured from porous materials, for example, polyolefin, polyester, nylon or cellulose acetate and can be obtained from Filtrona Inc. 8401 Jefferson Davis Highway, PO Box 34668, Richmond, Va. 23234, US. The sample collection device also includes a handhold (3) to facilitate manipulation by the patient when obtaining a sample and also to reduce the likelihood of contamination of the body fluid sample with, for example skin cells or bacteria which may affect a bioassay conducted on the sample once it has been processed. The handhold may be integral with the sample collection part. In this embodiment the handhold is manufactured from flexible plastic formed into a tube which fits about the sample collection part. The kit includes a scapula or similar skin puncture means. In this embodiment, the skin puncture means (4) is provided with actuation means to propel needle into the skin. This allows the patient to apply sufficient pressure to propel the needle to pierce the skin without penetrating too deeply to cause undue pain and discomfort. The kit is also provided with an antiseptic wipe impregnated or soaked with an antiseptic which is used to clean the wound of excess blood and prevent infection. The kit also provides a plaster to cover the wound to prevent further bleeding and protect to wound site from infection. The wick (2) is coated with a non-ionic surfactant, preferably a non-ionic detergent. Preferably said detergent is selected from Triton X100 or Tween 20. Typically the wick is treated with a solution of about 1% (v/v) detergent. The detergent coating facilitates the binding of the sample in the wick.

Sample Collection

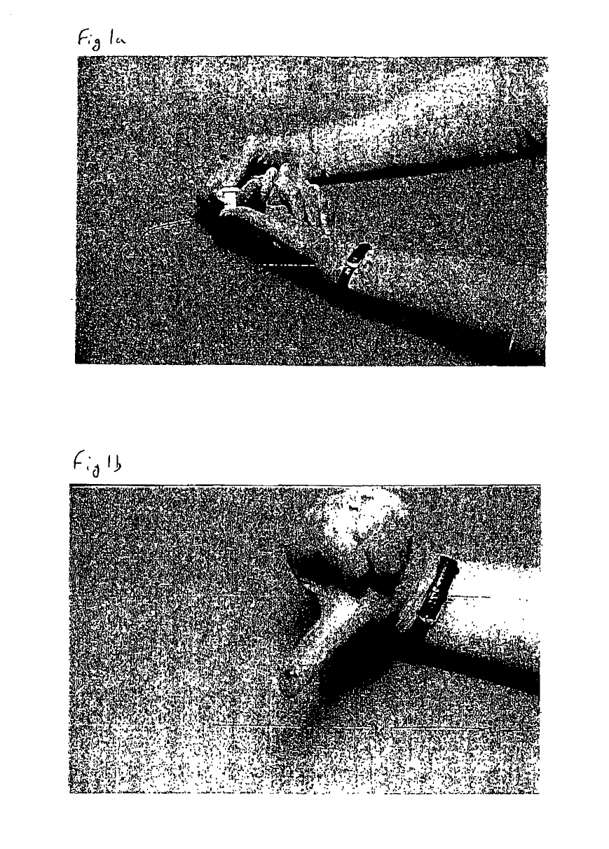

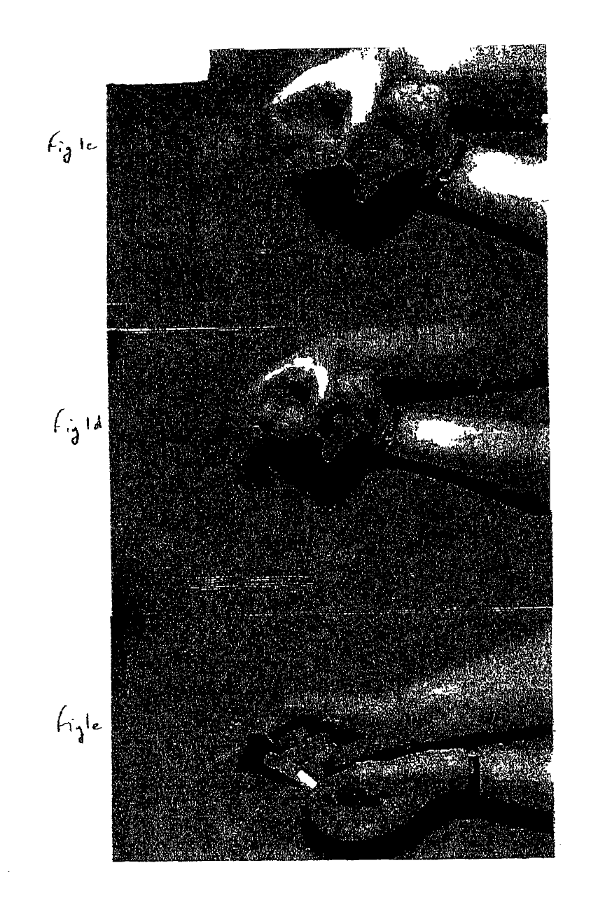

A blood sample is obtained from a patient by puncturing the thumb or finger with a lancet or similar device, FIG. 1a. This can be done either by the patient or by a practitioner. A small aliquot of blood, approximately 50-100 μl, accumulates on the outer surface of the thumb or finger. The patient or practitioner applies the sample collection part (2) to the blood sample. The porosity of the sample collection part facilitates sample collection by capillary action. The size of the sample collection part is dimensioned so that a sufficient sample is obtained, (i.e. approximately 50 μl). The sample collection part is applied to the blood sample, FIG. 1c, until the part is full of blood, FIG. 1d. The sample collection part is therefore adapted to provide volumetric sample collection.

This means that when a body fluid sample is brought into contact with the collection part capillary action fills the porous collection part. By providing a collection part of defined dimensions a sufficient sample can be obtained to allow a reliable test. Moreover the provision of a porous sample collection part, as opposed to a traditional capillary tube, means that the patient does not have to apply continuous contact to the sample to be collected to ensure an adequate sample is taken. Traditional capillary tubes, to be effective in sample collection, have to be in continuous contact with the sample.

The sample is allowed to dry in the sample collection part. The patient then places the saturated sample collection device into a suitable container, FIG. 1e. Typically the container is a screwed topped container, for example a cryovial (4). The patient can then forward the sample to a practitioner for subsequent processing of the sample from the collection device and bioassay.

Sample Processing

The extraction buffer typically includes a detergent, for example Tween 20 or Triton X100. A typical extraction buffer includes 2.7M NaCl, 50 mM KCl, 150 mM Na₂HPO₄, 30 mM KH₂PO₄, 0.05% (v/v) Tween 20 and 0.1% (w/v) polyvinyl pyrrolidone. The buffer should be sufficient to effectively extract the sample from the sample collection device but not include components which will interfere with the bioassay subsequently conducted on the sample. 10 ml of extraction buffer is placed in a suitable container, for example a scintillation vial. 1 ml of the extraction buffer is added to the cryovial containing the sample collection device. The cryovial is centrifuged at approximately 1000 g for 20 minutes, or until the blood sample is removed from the wick. The extracted sample is then removed from the cryovial and diluted in the remaining 9 ml of extraction buffer in the scintillation vial. The sample is then labelled and either assayed directly or stored, either refrigerated or frozen, prior to assay.

The present invention is not limited in scope by the examples provided, since the examples are intended as illustrations of various aspects of the invention and other functionally equivalent embodiments are within the scope of the invention. Various modifications of the invention in addition to those shown are described herein will become apparent to those skilled in the art for the foregoing description and fall within the scope of the appended claims. The advantages and objects of the invention are not necessarily encompassed by each embodiment of the invention.

All references, patents, and patent publications that are recited in this application are incorporated in their entirety herein by reference.

1. A method for the collection of body fluid samples comprising the steps of:
i) contacting a body fluid sample with a porous support material; and
ii) allowing said sample to dry to said porous support material.

2. A method according to claim 1, further comprising the step of:
storing said porous support material containing said sample.

3. A method according to claim 1, further comprising the step of:
extracting from said porous support material soluble components from insoluble cellular material retained in said porous support material.

4. A method according to claim 2, further comprising the step of:
extracting from said porous support material soluble components from insoluble cellular material retained in said porous support material.

5. A method according to claim 1 wherein said body fluid sample is selected from the group consisting of: blood; semen; lymph fluid; cerebral spinal fluid; synovial fluid; tears; sweat; urine; saliva; and bone marrow.

6. A method according to claim 1 wherein said porous support material is selected from the group consisting of: polyester; cellulose acetate; polyolefin; and nylon.

7. A method according to claim 6 wherein said porous support material is polyester.

8. A method according to claim 1 wherein said porous support material is coated with a non-ionic surfactant.

9. A method according to claim 8 wherein said non-ionic surfactant is a non-ionic detergent.

10. A method according to claim 9 wherein said non-ionic detergent is selected from the group consisting of Triton X100 and Tween 20.

11. A method according claim 8 wherein said porous support material is coated with at least about 0.05% w/w surfactant.

12. A method according to claim 8 wherein said porous support material is coated with no more than about 2.0% w/w surfactant.

13. A method according to claim 8 wherein said porous support material is coated with about 1% w/w surfactant.

14. A method according to claim 1 wherein said body fluid sample contains an immunoglobulin.

15. A method according to claim 14 wherein said immunoglobulin is selected from the following Ig isotypes: IgA, IgM, IgD, IgE or IgG.

16. A method according to claim 1 wherein said soluble components contains a metabolic intermediate.

17. A method according to claim 1 wherein the extraction of soluble components from said porous support material utilizes a high ionic strength extraction buffer.

18. A body fluid sample collection device comprising:
a porous sample collection part and associated therewith a handhold to facilitate sample collection.

19. A body fluid sample collection device according to claim 18 wherein said porous sample collection part is manufactured from at least one material selected from the group consisting of: polyester; cellulose acetate; polyolefin; and nylon.

20. A body fluid sample collection device according to claim 19 wherein said porous sample collection part is manufactured from polyester.

21. A body fluid sample collection device according to claim 18 wherein said porous sample collection part comprises an elongate member which serves as a wick to facilitate sample collection.

22. A body fluid sample collection device according to claim 21 wherein said porous sample collection part has a substantially circular cross-sectional shape.

23. A body fluid sample collection device according to claim 18 wherein said handhold is integral with said porous sample collection part.

24. A body fluid sample collection device according to claim 18 wherein said handhold is adapted to fit about said porous sample collection part.

25. A body fluid sample collection device according to claim 24 wherein said handhold comprises a member with a throughbore through which said sample collection part passes.

26. A body fluid sample collection device according to claim 18 wherein the porous sample collection part is dimensioned to obtain an aliquot of bodily fluid sufficient to conduct at least one assay.

27. A body fluid sample collection device according to claim 18 wherein the device is sterile.

28. A body fluid sample collection device according to claim 18 wherein said porous sample collection part is provided with a surfactant coating.

29. A body fluid sample collection device according to claim 28 wherein said surfactant coating is a detergent coating.

30. A body fluid sample collection device according to claim 29 wherein said detergent coating is provided by Triton X100 and/or Tween 20.

31. A body fluid sample collection kit comprising: a porous support material and a storage container for said porous support material.

32. A body fluid sample collection kit according to claim 31 further comprising a skin puncture device.

33. A body fluid sample collection kit according to claim 31 further comprising antiseptic wipes and/or adhesive dressing.

34. A body fluid sample collection kit according to claim 32 further comprising antiseptic wipes and/or adhesive dressing.

35. A body fluid sample collection kit according to claim 31 wherein said porous support material is a sample collection device comprising:
a porous sample collection part and associated therewith a handhold to facilitate sample collection.

36. A body fluid sample collection kit according to claim 31 wherein said skin puncture device is selected from the group consisting of a blade, a lancet and a needle.

37. A body fluid sample collection kit according to claim 31 wherein said skin puncture device is a needle which is provided with actuation means to propel the needle through the patient's skin.

38. A body fluid sample collection kit according to claim 37 wherein said storage container is a provided with a screw top cap.

39. A body fluid sample collection kit according to claim 38 wherein said screw top cap is provided with a seal to prevent contamination of the sample contained therein.

40. A body fluid sample collection body fluid sample collection kit according to claim 31 wherein the kit components are contained within a sealed container.
